(19) United States
(12) Patent Application Publication
(10) Pub. No.: US 2020/0146606 A1
(43) Pub. Date: May 14, 2020

(54) BODILY FLUID COLLECTION DEVICES INCLUDING ELECTRONICS AND ASSOCIATED SYSTEMS AND METHODS

(71) Applicant: Tasso, Inc., Seattle, WA (US)

(72) Inventors: Ben Casavant, Seattle, WA (US); Erwin Berthier, Seattle, WA (US)

(21) Appl. No.: 16/682,134

(22) Filed: Nov. 13, 2019

Related U.S. Application Data
(60) Provisional application No. 62/760,810, filed on Nov. 13, 2018.

(51) Int. Cl. A61B 5/15; A61B 5/1172; A61B 5/1171

(52) U.S. Cl. CPC A61B 5/150854; A61B 5/150099; A61B 5/150221; A61B 2560/0252; A61B 5/1172; A61B 5/1171; A61B 2560/0214; A61B 5/150824

(57) ABSTRACT

Bodily fluid withdrawing devices including electronics, and associated systems and methods, are disclosed. In some embodiments, the devices include a housing containing a bodily fluid withdrawing feature, an actuator movable relative to the housing, and one or more electronic component(s). The electronic component(s) can be configured to transition from an inactive state to an active state when the device is actuated and a circuit of the device is closed. Upon transitioning to the active state, the electronic component(s) can be configured to wirelessly transmit information and/or receive information from an external recipient. In some embodiments, the devices disclosed herein can comprise part of an interconnected system including one or more communication devices.

### CROSS-REFERENCE TO RELATED APPLICATION

This application claims the benefit of U.S. Patent Application No. 62/760,810, filed Nov. 13, 2018, and titled “BODILY FLUID COLLECTION DEVICES INCLUDING ELECTRONICS, AND ASSOCIATED SYSTEMS AND METHODS,” which is incorporated herein by reference in its entirety.

### TECHNICAL FIELD

The present technology is related to bodily fluid collection devices including electronics for determining characteristics of the collected body fluid, the collection device, the patient and/or other aspects of collecting body fluids. For example, various embodiments of the present technology are related to handheld bodily fluid collection devices configured to determine characteristics of withdrawn bodily fluid and wirelessly communicate with one or more external recipients.

### BACKGROUND

Conventional methods for withdrawing and analyzing bodily fluid (e.g., blood) from a patient or subject can be tedious and time consuming, often requiring the individual to travel to a clinic and then wait to receive results of the bodily fluid analysis. In clinical trials, for example, the process of obtaining a sample often requires the patient to travel to a clinic, verify identity, and then withdraw the sample. For some clinical trials, the patient is further required to take a substance (e.g., a chemical compound or placebo) and wait to withdraw the sample until after a predetermined amount of time so as to allow the substance to enter the patient's system.

In an effort to overcome these difficulties, a number of medical devices have been developed for patients to withdraw their own bodily fluid using a take-home, handheld device that is simple to operate. While these devices can be effective for obtaining a bodily fluid sample, they also create additional issues related to, e.g., quality control and chain-of-custody. As a result, there exists a need for improved techniques and systems for processes related to withdrawing bodily fluid from patients.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIG. 1A is a perspective view of a bodily fluid collection device configured in accordance with embodiments of the present technology.

FIG. 1B is a perspective view of the bodily fluid collection device of FIG. 1A in use.

FIG. 1C is a perspective view illustrating detachment of a collection reservoir from the bodily fluid collection device of FIG. 1A.

FIG. 2A is a partially schematic cross-sectional illustration of a bodily fluid collection device in an unactuated position and configured in accordance with embodiments of the present technology.

FIG. 2B is a partially schematic cross-sectional illustration of the bodily fluid collection device of FIG. 2A in an at least partially actuated position.

FIG. 2C is a schematic illustration of an electronics subsystem of FIG. 2A.

FIG. 2D is a top view of the bodily fluid collection device of FIG. 2A.

FIG. 3 is a block diagram of a method for operating the bodily fluid collection device shown in FIG. 2A in accordance with embodiments of the present technology.

FIG. 4A is a partially schematic perspective view illustrating a portion of the bodily fluid collection device shown in FIG. 2A, including a fluidic channel and sensor(s), and configured in accordance with embodiments of the present technology.

FIG. 4B is a partially schematic perspective view of the device shown in FIG. 4A in use.

FIG. 5 is a block diagram of a method for withdrawing bodily fluid and measuring characteristics associated with the withdrawn bodily fluid in accordance with embodiments of the present technology.

FIG. 6 is a block diagram of a method for measuring characteristics associated with withdrawn bodily fluid after administering a chemical compound in accordance with embodiments of the present technology.

FIG. 7A is a partially schematic cross-sectional illustration of a collection device including a collection reservoir and an unattached cap configured in accordance with embodiments of the present technology.

FIG. 7B is a partially schematic cross-sectional illustration of the collection device of FIG. 7A having the cap attached to the collection reservoir.

FIG. 7C is a partially-schematic top view of the cap 750 of FIG. 7A.

FIG. 7D is a partially schematic cross-sectional illustration of another embodiment of a collection device including a collection reservoir and an unattached cap configured in accordance with embodiments of the present technology.

FIG. 7E is a partially schematic cross-sectional illustration of the collection device of FIG. 7D having the cap attached to the collection reservoir.

FIG. 8 is a schematic illustration of a representative interconnected computing system including a medical device configured in accordance with embodiments of the present technology.

FIG. 9 is a partially schematic perspective view of a communication module configured in accordance with embodiments of the present technology.

FIG. 10 is a schematic illustration of representative screenshots of a system including a communication device used to communicate with the bodily fluid collection device of FIG. 2A in accordance with embodiments of the present technology.

FIG. 11 is a block diagram of a method for determining characteristics associated with withdrawn bodily fluid in accordance with embodiments of the present technology.

FIG. 12 is a block diagram of a method for determining characteristics associated with withdrawn bodily fluid, including an identity verification step, in accordance with embodiments of the present technology.

### DETAILED DESCRIPTION

### I. Overview

Aspects of the present disclosure are directed generally to devices, systems, and methods for withdrawing and/or collecting bodily fluid from a patient, and determining characteristics of the bodily fluid, the patient, the collection device and/or other aspects of collecting a bodily fluid. For example, some embodiments of the present technology are directed to a bodily fluid collection device having a housing containing a bodily fluid withdrawing feature, an actuator movable relative to the housing, and one or more electronic component(s). The electronic component(s) can be activated by actuating the device, or more particularly, by moving the actuator to an at least partially actuated position, thereby causing a movable switch to close a circuit of the device. The electronic component(s) can include one or more sensor(s) or measurement components for measuring temperature, elapsed time, geographic location, patient identity, device orientation (e.g., direction the device is facing), and/or chemical compounds. The device can further include a battery, a computer-readable medium having instructions, and a processor configured to execute the instructions. The electronic component(s) and processor may be operably coupled to the battery when the circuit is closed.

Embodiments of the present technology are also related to a collection device (e.g., a reservoir) for collecting bodily fluid from a patient and determining characteristics of the bodily fluid. The bodily fluid collection devices can be part of a computing system including, for example, a user device (e.g., a mobile device), an external server, and/or a communication module. Each of the user device, external server and communication module can interact with the bodily fluid collection devices. For example, each of the user device, external server and communication module can be configured to receive information associated with the bodily fluid collection device or bodily fluid withdrawn therefrom.

In one aspect of the present technology, a bodily fluid collection device can include one or more electronic components that enable the devices to determine and store characteristics of a withdrawn bodily fluid sample. For example, the bodily fluid collection devices can measure, diagnose, monitor, record, verify, analyze, assess, communicate, etc., characteristics of the withdrawn body fluid, the patient, the environment, the fluid collection device and/or other aspects of the technology and the related process. In some embodiments, for example, the devices can determine and store a temperature of the device or bodily fluid sample, and a time associated with when the bodily fluid sample was withdrawn from the patient. In doing so, embodiments of the present technology can provide improved methods for withdrawing a bodily fluid sample, while also ensuring quality control of the sample at least because characteristics of the sample can be monitored on a continuous basis from the time a sample is withdrawn. Furthermore, the electronic component(s) of the bodily fluid collection devices can enable the devices to form part of a computing system in which information corresponding to the stored characteristics of the bodily fluid sample can be sent to external recipients, such as a user device, database, or server. For example, the communication module can be used to transport the bodily fluid sample while also tracking, e.g., temperature in the communication module. In addition to or in lieu of the foregoing, the user device and/or communication module can be used to verify an identity of the patient or bodily fluid collection device and provide an electronic record of chain-of-custody. For example, the communication module can be configured to track a geographic location of the container and when the container is open or closed (i.e., when the sample is potentially tampered with). As such, embodiments of the present technology can provide systems and associated methods for establishing improved chain-of-custody accountability for the sample between the time the sample is withdrawn from the patient to when the sample reaches the laboratory or final destination.

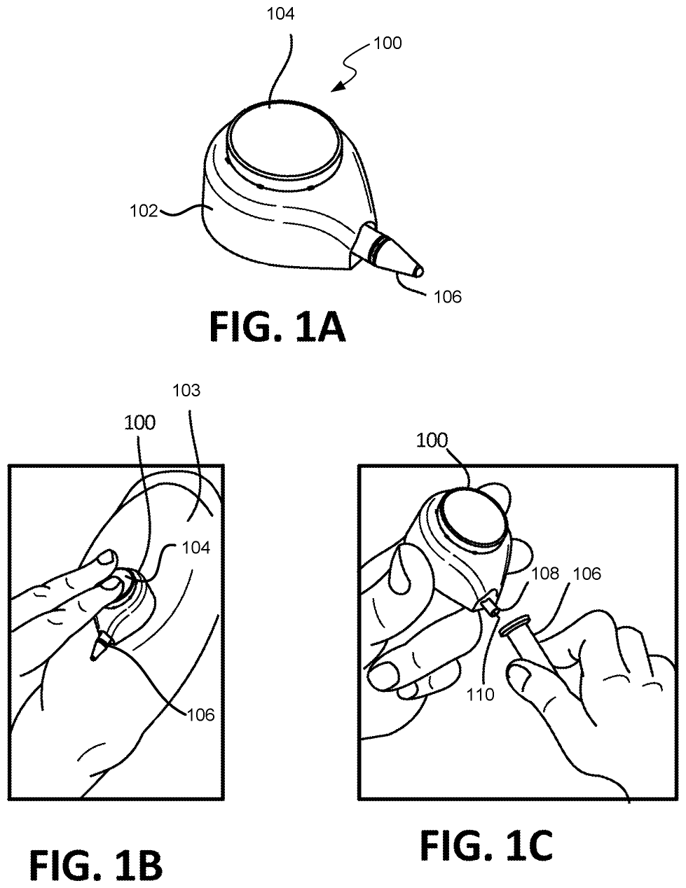

Specific details of the present technology are described herein with reference to FIGS. 1A-12. It should be noted that while many of the embodiments are described below with respect to devices, systems, and methods for withdrawing bodily fluid samples from patients for individual diagnosis, other applications and other embodiments in addition to those disclosed herein are within the scope of the present technology. For example, the devices and computing systems of the present technology may be used for clinical trials in the biotech or pharmaceutical industries, as well for other industries in which verifying patient identity of a bodily sample is important (e.g., life insurance). In addition, some embodiments of the present technology can have different configurations, components, and/or procedures than those shown or described herein. Moreover, a person of ordinary skill in the art will understand that some embodiments of the present technology can have configurations, components, and/or procedures in addition to those shown or described herein and that these and other embodiments can be without several of the configurations, components, and/or procedures shown or described herein without deviating from the present technology.

The headings provided herein are for convenience only and should not be construed as limiting the subject matter disclosed.

### II. Bodily Fluid Collection Devices

FIG. 1A is a perspective view of a bodily fluid collection device 100 configured in accordance with embodiments of the present technology. The device 100 can be handheld with a size easily grasped and manipulated by one or both of a patient's hands. Such handheld devices advantageously allow a patient to collect a bodily fluid sample (e.g., a blood sample) without assistance from another individual. The handheld devices of the present technology can be operated by a layperson outside of a medical setting (e.g., at home or in a field clinic) and without aid of a medical professional (e.g., a physician or nurse).

As shown in FIG. 1A, the device 100 includes a housing 102 and an actuator 104 movable relative to the housing. The actuator 104 (e.g., a lid or button) effectuates withdrawal of a bodily fluid from the patient. The housing 102 is removably coupled to a collection reservoir 106 (e.g., a tube or cartridge) for receiving the bodily fluid withdrawn from the patient. The reservoir 106 can act as a removable and standardized container for bodily fluid that can be detached and inserted into clinical and laboratory equipment or workflows, e.g., for diagnostics and/or biomarker detection.

FIG. 1B is a perspective view of the device 100 in use. To collect a bodily fluid sample, a bottom surface of the housing 102 is pressed against the patient's skin 103 such that the actuator 104 is positioned away from the skin 103. Pressing the actuator 104 deploys a skin-piercing feature (e.g., a lancet, blade, or needle) from within the device 100 to pierce the skin 103. Subsequent retraction of the actuator 104 away from the skin can create a vacuum within the device 100 that acts against the skin 103 either directly or indirectly. The vacuum draws bodily fluid from the resulting incision into the housing 102 where it is then channeled to the reservoir 106.

FIG. 1C is a perspective view illustrating detachment of the reservoir 106 from the device 100. After pressing the actuator 104 and piercing the skin 103, bodily fluid is withdrawn from the patient into a microfluidic network having one or more channels 110 that direct the bodily fluid from a collection site to the reservoir 106. Once the desired amount of bodily fluid is in the reservoir 106, the device 100 can be removed from the skin 103 and the reservoir 106 detached from the housing 102, e.g., by decoupling the reservoir 106 from a coupling portion 108 of the device 100.

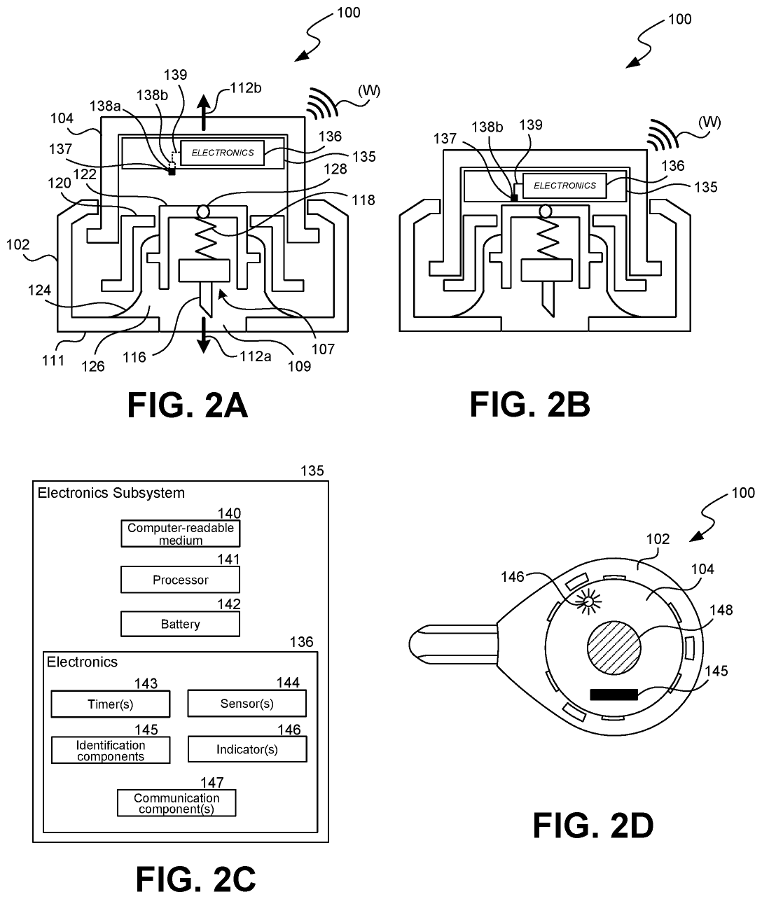

FIG. 2A is a partially schematic cross-sectional illustration of the device 100 in an unactuated position, and FIG. 2B is a partially schematic cross-sectional illustration of the device 100 in a partially actuated position in accordance with embodiments of the present technology. Referring first to FIG. 2A, the device 100 includes the housing 102, the actuator 104, a skin-piercing assembly 107 located at least partially or completely within the housing 102, and an opening 109 extending through the housing 102. In some embodiments, the opening 109 is formed in a bottom surface 111 of the housing 102 such that the opening 109 is at the skin when the device 100 is applied to a patient's body. The actuator 104 is movable relative to the housing 102 between an unactuated position (as shown in FIG. 2A) to an actuated position (as shown in FIG. 2B) along a deployment direction 112a and a retraction direction 112b. The deployment direction 112a can be a downward direction in the orientation of FIG. 2A, e.g., towards the opening 109, and the retraction direction 112b can be an upward direction in the orientation of FIG. 2A, e.g., away from the opening 109. As such, the deployment direction 112a is generally toward the skin, while the retraction direction 112b is generally away from the skin.

The skin-piercing assembly 107 includes at least one skin-piercing feature 116 (e.g., a lancet, blade, or needle) and a biasing member 118 (e.g., a spring) that is coupled to the skin-piercing feature 116. The biasing member 118 is configured to drive the skin-piercing feature 116 along the deployment direction 112a towards the opening 109. The skin-piercing feature 116 can be configured to pierce the patient's skin to create an incision from which bodily fluid can be withdrawn. The size of the skin-piercing feature can be varied as desired. For example, a relatively large skin-piercing feature can be advantageous for creating a larger incision that allows for withdrawal of larger volumes of bodily fluid. A relatively small skin-piercing feature can be advantageous for reducing pain and achieving high penetration velocities. Optionally, the skin-piercing assembly 107 can include a plurality of skin-piercing features, e.g., two, three, four, five, or more skin-piercing features. In some embodiments, the device 100 can include a corresponding number of openings 109, such that each skin-piercing feature passes through a respective opening to pierce the patient's skin. However, in some embodiments, more than one skin piercing feature 116 can pass through an opening 109. For example, all of the skin piercing features 116 can pass through a single opening 109.

Still referring to FIG. 2A, the device 100 includes an actuation mechanism for actuating the deployment of the skin-piercing feature 116. For example, the actuator 104 can be mechanically coupled to the skin-piercing assembly 107, e.g., via a platform 120 and a plunger 122, to deploy the skin-piercing feature 116 along the deployment direction 112a. In the illustrated embodiment, the platform 120 is located at least partially within the actuator 104, the plunger 122 is located at least partially within the platform 120, and the skin-piercing assembly 107 is coupled to the plunger 122. Optionally, the actuation mechanism can also actuate the retraction of the skin-piercing feature 116 along the retraction direction 112b, after the skin-piercing feature 116 has been deployed.

In some embodiments, the device 100 further includes a vacuum mechanism to facilitate collection of the bodily fluid. For example, the device 100 can include a sealing member 124 (e.g., a flexible membrane) that can bend and/or is elastic, and is over the opening 109 to form a lumen 126. The device 100 can include at least one valve 128 fluidically connected to the lumen 126 to control air flow into and out of the lumen 126. The sealing member 124 can be mechanically coupled to the skin-piercing assembly 107, e.g., via the plunger 122, such that movement of the skin-piercing assembly 107 along the deployment direction 112a decreases the volume of the lumen 126, and movement of the skin-piercing assembly 107 along the retraction direction 112b increases the volume of the lumen 126. The valve 128 can be a one-way valve that permits air to escape from within the lumen 126, e.g., as the lumen volume decreases, but prevents air from entering the lumen 126, e.g., as the lumen volume increases. This creates a low-pressure region (e.g., a vacuum) within the lumen 126 that acts directly or indirectly against the patient's skin. Additional features and implementations of the device 100, including the actuation mechanism, vacuum mechanism, and other features are described in additional detail with reference to U.S. Provisional application Ser. No. 16/571,028, entitled “Bodily Fluid Collection Devices and Related Methods, filed Sep. 13, 2019,” the disclosure of which is incorporated herein by reference in its entirety.

Referring next to FIGS. 2A and 2B together, the device 100 can include an electronics subsystem 135 having circuitry 139 (e.g., conductive traces), one or more switch(es) 137 (referred to herein as “the switch 137”), and one or more electronic component(s) 136. As shown in FIG. 2C, the electronics subsystem 135 can further include a non-transitory computer-readable medium 140 storing (e.g., having, containing, etc.) instructions, a processor 141 configured to execute the instructions, and one or more batteries 142 (referred to as “the battery 142”). The computer-readable medium 140 can include non-volatile memory and/or volatile memory for storing information (e.g., about the bodily fluid) and/or executing a database. The electronic component(s) 136 can include one or more timer(s) 143 (e.g., a clock), sensor(s) 144, identification component(s) 145, indicator(s) 146 (e.g., light indicators, audible indicators, etc.) and communication components 147 (e.g., transmitters, receivers, and associated communication circuitry). The communication components 147 can be used to send and/or receive wireless messages (W), e.g., via one or more proprietary or standardized wireless standards, such as 802.11, Bluetooth, near-field communication (NFC) and/or other similar communication means. In some embodiments, the communication components 147 can communicate with one or more remote computing devices (e.g., a remote server) via a network, such as a local area network (LAN), a personal area network (PAN), a wide area network (WAN), and/or a combination of such networks. The network may be the Internet or some other public or private network.

In general, the present technology (e.g., the computer-readable medium 140 and/or the electronic components 136) can be embodied as special-purpose hardware (e.g., circuitry), as programmable circuitry appropriately programmed with software and/or firmware, or as a combination of special-purpose and programmable circuitry. Hence, embodiments of the present technology can include a machine-readable medium having stored thereon instructions which may be used to cause a computer, a microprocessor, processor, and/or microcontroller (or other electronic devices) to perform a process. Specifically, for example, the non-transitory computer-readable medium 140 can store instructions that, when executed by the one or more processors 141, cause the electronics subsystem 135 to perform any of the actions, operations, methods, etc., described in detail herein. The machine-readable medium may include, but is not limited to, optical disks, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions.

The present technology can also be practiced in distributed computing environments, where tasks or modules are performed by remote processing devices, which are linked through a communications network, such as a LAN, WAN, or the Internet. In a distributed computing environment, program modules or sub-routines may be located in both local and remote memory storage devices. Aspects of the technology described above may be stored or distributed on computer-readable media or, alternatively, aspects of the technology may be distributed electronically over the Internet or over other networks (including wireless networks). Those skilled in the relevant art will recognize that portions of the technology may reside on a server computer, while corresponding portions reside on a client/user computer. Data structures and transmission of data particular to aspects of the technology are also encompassed within the scope of the technology.

Referring again to FIGS. 2A and 2B, the electronic component(s) 136 are within or on the actuator 104. In other embodiments, the electronic component(s) 136 may be positioned elsewhere on the device 100, such as within the housing 102. In addition to or in lieu of the foregoing, the electronic component(s) 136 may be positioned separate from the circuitry 139 and/or switch 137.

The battery 142 can include lithium (e.g., lithium ion or lithium polymer), nickel, alkaline, a combination thereof, or other materials commonly used in batteries for handheld medical devices. The battery 142 can be operably coupled to the electronic component(s) 136 to provide power thereto. In some embodiments, a single battery may be used to power all of the electronic component(s) 136 on the device 100, whereas in other embodiments, individual batteries may be used to power individual electronic component(s) 136. For example, in some embodiments, the device 100 can include a first battery operably coupled to the communication components 147, and one or more second batteries operably coupled to the other electronics. In such embodiments, the first battery may enable the device 100 to transmit and/or receive a wireless signal (E) information at all times (e.g., even prior to actuation of the device 100), whereas the second batteries may only be activated after the device is actuated, thereby saving battery life of the second batteries until after the device 100 has been actuated and a bodily fluid sample has been withdrawn from the patient. As described in additional detail below, one advantage of such embodiments is extending the amount of time the bodily fluid can be monitored, e.g., via the sensor(s).

The switch 137 can include a metal or another conductive material (e.g., copper, aluminum, alloys thereof, graphite, thermally-conductive polymers, etc.). The switch 137 can be actuated via pressure and, as shown in the illustrated embodiment, moved from a first position 138a to a second position 138b. More specifically, as the actuator 104 is pressed and moved in the deployment direction 112a toward the platform 120, the platform 120 forces the switch 137 from the first position 138a to the second position 138b. In the first position 138a, a circuit associated with the switch 137 is open, and in the second position 138b the circuit is closed. The circuit can include the switch 137, circuitry 139, battery 142 and at least a portion of the electronic component(s) 136. In some embodiments, the switch 137 is not moved back to the first position 138a when the actuator is released, thereby maintaining the circuit in the closed position. Closing the circuit can thus activate the electronic component(s) 136 and transition from an inactive state to an active state. Such a transition may occur only after the device 100 is actuated and/or bodily fluid is withdrawn from the patient. By activating certain electronic component(s) 136 only after the device 100 is actuated, battery life is prolonged compared to maintaining the electronic component(s) 136 in the active state. In some embodiments, for example, the electronic component(s) 136 can be energized via a battery for at least 12 hours, 24 hours, 48 hours, 3 days, 4 days, 5 days, 6 days, 7 days, or more, while transmitting and/or logging information of measured characteristics at predetermined intervals (e.g., 10 minutes, 30 minutes, 1 hour, 2 hours, 5 hours, etc.).

Once activated, the electronic component(s) 136, such as the sensor(s) 144, can measure and/or store characteristics of the bodily fluid via the computer-readable medium 140. In addition to or in lieu of the foregoing, the electronic component(s) 136 can also wirelessly transmit and/or receive information associated with the characteristics to an external device or recipient (e.g., a database, mobile device, server, etc.).

The sensor(s) 144 can include analyzers or other components for measuring characteristics of the device 100 or bodily fluid. For example the sensor(s) 144 can be configured to measure temperature (e.g., of the bodily fluid or in the device), time (e.g., time of day or elapsed time), humidity, geographic location, accelerometer data (e.g., to detect device movement or device diagnostics), device orientation, amount of bodily fluid collected (e.g., in the reservoir), interior pressure (e.g., amount of vacuum after actuation, force pressure (e.g., of actuator) heart rate (e.g., via a light sensor), blood pressure (e.g., via an infrared (IR) sensor), and/or patient identification (e.g., fingerprint analysis). In addition to or in lieu of the foregoing, the sensor(s) can be configured to measure characteristics of the bodily fluid, including metabolites, biologics, biomarkers, proteins, platelets, antibodies, RNA (e.g., short hairpin RNA), hydrophobic molecules, hydrophilic molecules, vitamins, diet markers, and/or chemical compounds (e.g., orally-ingestible chemical compounds that cross the mucosal or gut lining). For example, in some embodiments in which a patient's cholesterol levels are of interest, the sensor(s) can be configured to measure cholesterol, high-density lipoproteins, low-density lipoproteins, and/or triglycerides.

FIG. 2D is a top view of the device 100. As shown in the illustrated embodiment, the device 100 can include a sensor 148 for identifying a patient (e.g., a fingerprint sensor), one of the identification component(s) 147 for identifying the particular device, and one of the indicators 146. The patient-identifying sensor 148, device-identifying component 145, and indicator 146 can be positioned at an outer surface of the device 100, such as on the actuator 104, as shown in FIG. 2D. In some embodiments, the identifying sensor 148, identification component 145, and indicator 146 may be positioned elsewhere on the device 100, such as the housing 102. The patient-identifying sensor 148, identification component 145, and indicator 146 can be operably coupled to the one or more batteries, processor, and computer-readable medium previously described.

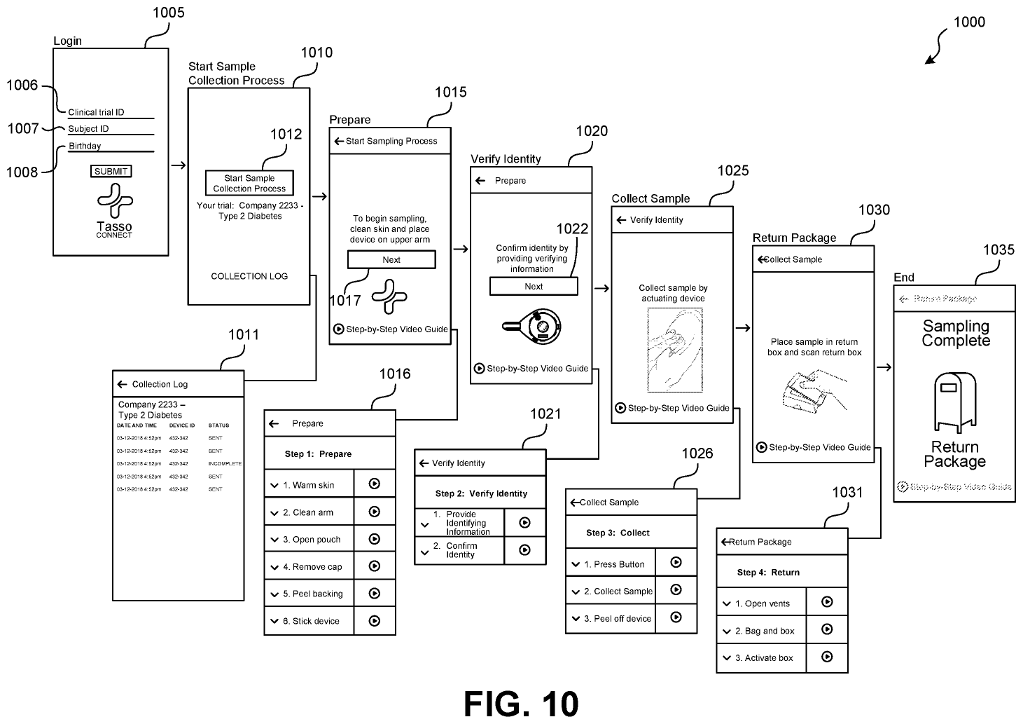

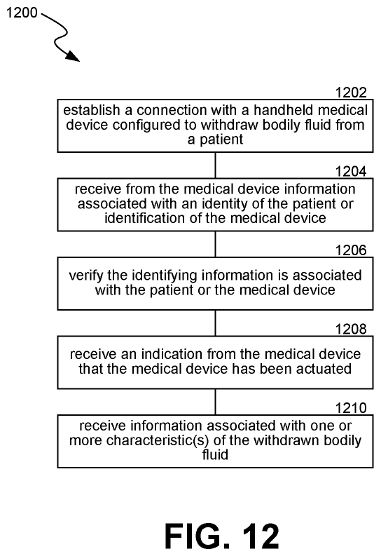

The patient-identifying sensor 148 can be used to determine or record an identity of a patient before or after the device 100 is actuated and bodily fluid is withdrawn. As described in more detail below (e.g., with reference to FIGS. 10 and 12, the patient-identifying sensor 148 can link the device 100 and/or the bodily fluid sample to a particular patient. This can help ensure the bodily fluid sample is not mistakenly assigned to another patient in later processing of the sample. As an example, a patient can scan their fingerprint via the patient-identifying sensor 148 to enable the device 100 to obtain patient-identifying information. The patient-identifying information can then be stored on the computer-readable medium 140 and be transmitted to an external recipient, where the patient-identifying information can be verified. In some embodiments, the device 100 can be configured to receive an indication that the patient-identifying information has been verified.

The identification component 145 can include a chip or tag configured for automatic identification and data capture. For example, the identification component 145 can include integrated circuits or components to enable radio-frequency identification (RFID) or optical recognition. The identification component 145 can be used to identify the particular device 100, such as a serial number of the device 100, and link the device 100 and/or bodily fluid sample taken therefrom to a particular patient, e.g., based on information in an external database. As described in more detail below with reference to FIGS. 10 and 12, the identification component 200 can be scanned or read by an external communication device, such as a user device (e.g., a patient's mobile device), a clinician's device, or a communication module, to obtain identifying information of the device 100. In some embodiments, the identifying information can then be used to link the particular device to the patient. In addition to or in lieu of the foregoing, information associated with the link established between the particular device and patient can be sent to and stored on the computer-readable medium of the device 100.

The indicator 146 can include a visible indicator (e.g., a light emitting diode) and/or an audible indicator (e.g., a speaker). The indicator 146 can be operably coupled to the computer-readable medium 140 of the device 100 and be programmed to produce different outputs based on particular actions of the device 100. The outputs can include a display of different colors, blinking a predetermined amount of times, or a combination thereof. The particular actions of the device that may produce the outputs can include, for example, a successful withdrawal of a bodily fluid sample, an established connection with an external device, identify verification of the device or patient, a low battery, or the like. In a particular embodiment, the indicator 146 may display (a) a green output for a successful withdrawal of bodily fluid, (b) a blinking green output (e.g., two blinks) for an established connection with an external device, (c) a blinking green output (e.g., three blinks) for a verification of identify, and (d) a red light for a low battery.

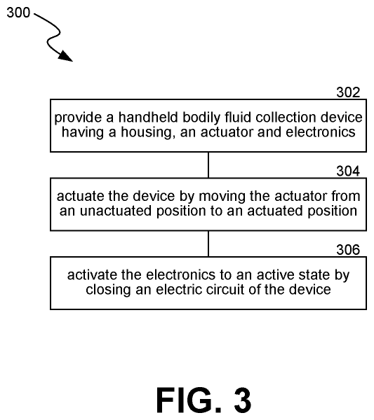

FIG. 3 is a block diagram of a method 300 for operating the device 100 shown in FIG. 2A in accordance with embodiments of the present technology. As shown in the illustrated embodiment, the method 300 includes providing a handheld bodily collection device having a housing, an actuator movable relative to the housing, and one or more electronic component(s) (process portion 302). The housing can include one or more bodily fluid withdrawing features configured to cause bodily fluid to be withdrawn from a patient. The method 300 can further comprise actuating the device by moving the actuator from an unactuated position to an actuated position (process portion 304), and after actuating the device, activating the electronic component(s) to an active state by closing a circuit of the device (process portion 306). In some embodiments, actuating the device can cause a switch to move from a first position to a second position, thereby closing the circuit and transitioning the electronics from an inactive state to the active state. In some embodiments, the switch can include magnetic components, and therefore moving the switch from the first position to the second position can include magnetically moving the switch from the first position to the second position.

In some embodiments, activating the electronic component(s) can include operably coupling a battery of the device to one or more of a processor, a timer or timing system, a sensor, an identification component, an indicator, and/or other components to provide communication capabilities (e.g., a transmitter, receiver, etc.). Operably coupling the battery to these electronic component(s) can activate them from the inactive state to the active state. As an example, the timer or timing system can be configured to monitor time elapsed since activating the electronic component(s), and closing the circuit can cause the processor to execute instructions of a computer-readable medium of the device to cause the device to measure a duration of time elapsed since the device was actuated. As another example, the communication components can enable the device to wirelessly transmit and/or receive information associated with the device or withdrawn bodily fluid, and closing the circuit can cause the processor to execute the instructions to cause the device to wirelessly transmit the information to an external recipient.

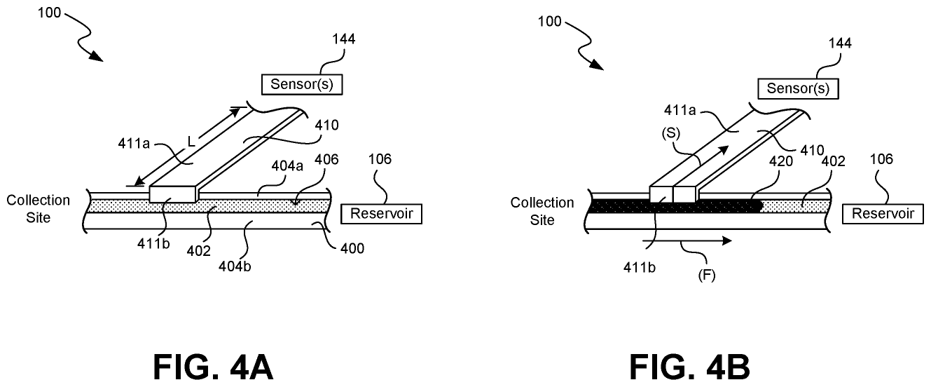

FIG. 4A is a partially schematic perspective view illustrating a portion of the device 100 shown in FIG. 2A, including a fluidic channel and sensor(s), in accordance with embodiments of the present technology. As previously described, bodily fluid can be withdrawn from the patient into a microfluidic network of the device 100 having one or more channel(s). The channel(s) can include one or more microfluidic channel(s) 400 (referred to as “the channel 400”) for directing the bodily fluid from a collection site toward the reservoir 106. As shown in the illustrated embodiment, the channel 400 can include a base surface 402, a pair of sidewalls 404a, 404b generally normal to and extending from the base surface 402, and an open end portion 406 extending over the base surface along a length of the channel 400. Various aspects of the channel 400 are described in additional detail in (a) U.S. application Ser. No. 13/949,108, filed Jul. 23, 2013, entitled “Methods, Systems, and Devices Relating to Open Microfluidic Channels”, and (b) U.S. application Ser. No. 14/816,994, filed Aug. 3, 2015, entitled “Devices, Systems and Methods for Gravity-Enhanced Microfluidic Collection, Handling and Transferring of Fluids”, each of which is incorporated herein by reference in their entireties.

As is also shown in the illustrated embodiment, the device 100 can include at least one of the sensors 144 previously described, and a sample board 410 extending at least partially between the channel 400 and the sensors 144. The sample board 410 can comprise a printed circuit board (PCB) or similar material configured to include conductive traces, and/or a polymer or plastic (e.g., Pyralux®, polyether ether ketone (PEEK), polyamide, etc.). In some embodiments, the sample board 410 can include a microfluidic channel or a fibrous substrate. Furthermore, the sample board 410 can include a first portion 411a connected to the sensors 144, and a second portion 411b attached to the first portion 411a and configured (e.g., positioned) to directly contact bodily fluid withdrawn and flowing along the channel 400. The first portion 411a can have a length (L), and can be generally flat or include a slight ramp (e.g., a height adjustment) between the sensors 144 and second portion 411b. In some embodiments, the length (L) and/or height adjustment can be varied depending on the chemistry or sensor(s) used for the detection of the bodily fluid and/or particular characteristic(s) of the bodily fluid (e.g., the particular analytes) to be measured by the sensors 144. For example, a longer length and/or larger height adjustment may be used to specifically filter and/or prevent certain chemical compounds from reaching the sensor(s) 144.

FIG. 4B is a partially schematic perspective view of the device shown in FIG. 4A in use. As shown in the illustrated embodiment, bodily fluid 420 flows in a direction (F) from the collection site over the base surface 402 of the channel 400 toward the reservoir 106. As the bodily fluid 420 flows in the channel 400, a portion of the bodily fluid contacts the second portion 411b of the sample board 410 and is drawn along the first portion 411a in a direction (S) toward the sensors 144. The bodily fluid 420 that reaches the sensors 144 can be analyzed for certain characteristics. In some embodiments, the analyzed characteristic(s) can be stored in the computer-readable medium of the device 100 and be transmitted to an external recipient, as previously described.

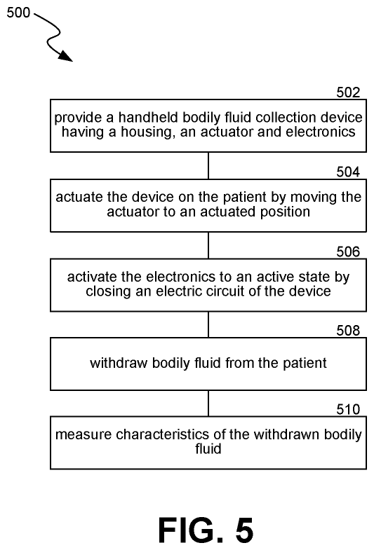

FIG. 5 is a block diagram of a method 500 for withdrawing a bodily fluid and measuring characteristics associated with the withdrawn bodily fluid in accordance with embodiments of the present technology. As shown in the illustrated embodiment, the method 500 is generally similar to the method 300 previously described. For example, the method 500 includes providing a handheld bodily fluid collection device having a housing, an actuator, and electronics (process portion 502), actuating the device on the patient by moving the actuator to an actuated position (process portion 504), and activating the electronics to an active state by closing a circuit of the device (process portion 506). The method 500 can further include withdrawing bodily fluid from the patient (process portion 508) and measuring characteristics of the withdrawn bodily fluid (process portion 510). Measuring the characteristics of the withdrawn bodily fluid is performed by one or more sensors on the device itself. A previously discussed, the measured characteristics of the bodily fluid can include metabolites, biologics, biomarkers, proteins, platelets, and/or chemical compounds.

At least one advantage of embodiments of the present technology described in FIGS. 2A-5 is the ability of a single device to withdraw a bodily fluid, and measure, record, send and/or receive information associated with the bodily fluid, the device, the patient and/or the environment. In doing so, the bodily fluid is measured immediately after being withdrawn, thereby limiting the likelihood that the sample is contaminated prior to being measured. In some embodiments, these initial measurements can act as a baseline measurement and be compared to subsequent measurements performed in a laboratory of the same sample. Yet another advantage of embodiments of the present technology is that by measuring the sample immediately after being withdrawn, a patient can receive feedback about the sample after just a short period of time (e.g., less than 10 minutes). Additionally, embodiments of the present technology simplify the often tedious conventional process associated with withdrawing and analyzing bodily fluid from a patient.

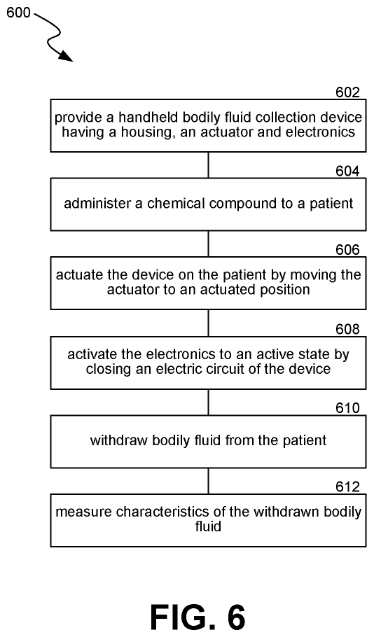

FIG. 6 is a block diagram of a method 600 for measuring characteristics associated with withdrawn bodily fluid after administering a chemical compound to a patient in accordance with embodiments of the present technology. The method 600 includes providing a handheld bodily fluid collection device having a housing, an actuator, and electronics (process portion 602), and administering a chemical compound to a patient (process portion 604). The method 600 further includes, after administering the chemical compound, actuating the device on the patient by moving the actuator to an actuated position (process portion 606). In some embodiments, actuating the device can be performed after a predetermined amount of time has elapsed since the chemical compound was administered. The predetermined amount of time may be determined based on the rate the chemical compound is expected to enter the patient's blood stream. The method 600 can further include activating the electronics to an active state by closing a circuit of the device (process portion 608), withdrawing bodily fluid from the patient (process portion 610), and measuring characteristics of the withdrawn bodily fluid (process portion 612). Measuring characteristics of the withdrawn bodily fluid can include measuring an amount of the chemical compound in the bodily fluid. In some embodiments, the measured amount of the chemical compound can then be compared to an expected amount of chemical compound that should be in the bodily fluid. For example, a measured amount of a drug taken by a patient can be compared to the standard clearance rate of the drug from the trial. If the measured amount of the drug is abnormal (e.g., above or below an expected range), then additional action may be taken, such as increasing or decreasing the prescribed amount of drug to be taken to match the standard clearance rate of the patient.

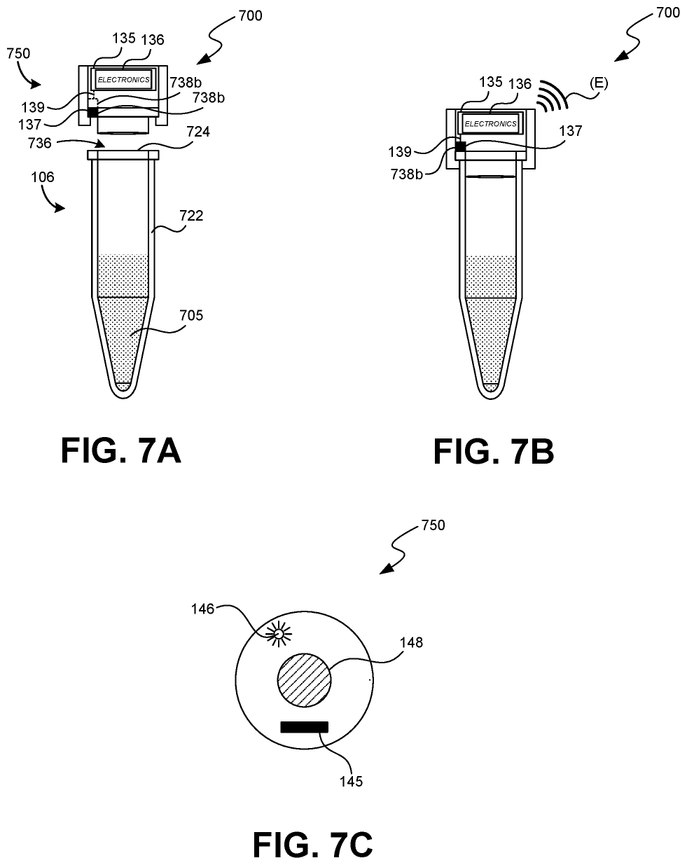

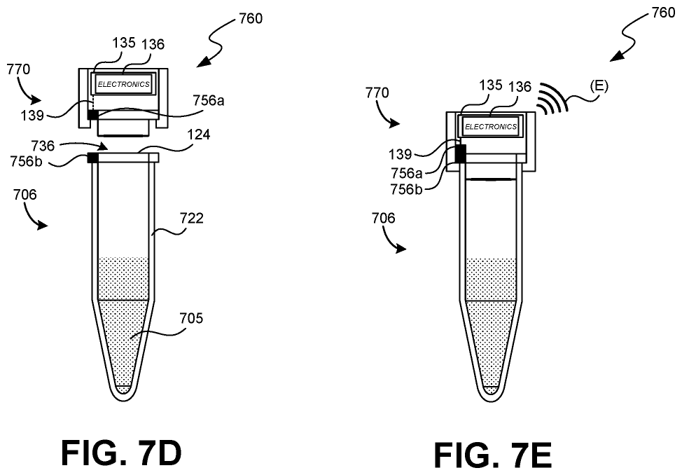

As previously described, bodily fluid withdrawn via the device 100 is directed to a collection reservoir 106 (FIGS. 1A-1C). FIG. 7A is a partially schematic cross-sectional illustration of a collection device 700 including the collection reservoir 106 previously described and an unattached cap 750, and FIG. 7B is a partially schematic cross-sectional illustration of the collection device 700 including the reservoir 106 with the cap 750 attached thereto in accordance with embodiments of the present technology. Referring first to FIG. 7A, the reservoir 106 is configured to hold a bodily fluid 705 (e.g., blood) and can include a sidewall 722, a platform 724 attached to the sidewall 722, and an open end portion 736 defined by the platform 724. The open end portion 736 can be sized to receive a portion of the cap 750. The cap 750 can be attached to the reservoir 106 via friction, a sealing mechanism, or other attachment means configured to prevent bodily fluid from escaping from the reservoir 106.

The cap 750 can include the electronics subsystem 135 previously described with reference to FIGS. 2A-2D, including the electronic component(s) 136, circuitry 139, and switch 137. Accordingly, the cap 750 can include the computer-readable medium 140 having instructions, processor 141 configured to execute the instructions, and battery 142. Furthermore, the electronic component(s) 136 can include one or more of the timers 143, sensors 144, identification components 145, indicators 146 and communication components 147, as previously described.

Referring to FIGS. 7A and 7B together, the switch 137 can be actuated via pressure and, as shown in the illustrated embodiment, be moved from a first position 738a to a second position 738b. More specifically, as the cap 750 is pressed onto the reservoir 106 toward the platform 724, the platform 724 forces the switch 137 from the first position 738a to the second position 738b. In the first position 738a, a circuit associated with the switch 137 is open, and in the second position 738b, the circuit is closed. The circuit can include the switch 137, the circuitry 139 and at least a portion of the electronic component(s) 136. Accordingly, in some embodiments, at least some of the electronic component(s) 136 may exist in an inactive state, and transition to an active state only after the switch 137 is moved to the second position 738b (i.e., when the reservoir 106 is attached to the cap 150). Once activated, the electronic component(s) 136, such as the sensors, can begin measuring and/or storing characteristics of the bodily fluid via the computer-readable medium, and wirelessly transmitting the measured and/or stored characteristics to an external recipient (e.g., a database, mobile device, server, etc.). By transitioning certain electronic component(s) 136 to their active state only after the collection device 700 is actuated, battery life is prolonged compared to if the electronic component(s) 136 were always in the active state. As previously described, the electronic component(s) 136 can be energized via the battery for at least 12 hours, 24 hours, 48 hours, 3 days, 4 days, 5 days, 6 days, 7 days, or more, while transmitting and/or logging information of the measured and/or stored characteristics at predetermined intervals (e.g., 10 minutes, 30 minutes, 1 hour, 2 hours, 5 hours, etc.).

FIG. 7C is a partially-schematic top view of the cap 750 of FIG. 7A. As shown in the illustrated embodiment, the collection device 700 can include the sensor 148 for identifying a patient (e.g., a fingerprint sensor), the identification component 145 for identifying the particular device, and the indicator 146, as previously described with reference to FIG. 2D. Each of the patient-identifying sensor 148, device-identifying component 145, and indicator 146 can be positioned at an outer surface of the collection device 300, such as on the cap 150, as shown in FIG. 2C. In some embodiments, the identifying sensor 148, identification component 145, and indicator 146 may be positioned elsewhere on the collection device 700, such as the reservoir 106. The identifying sensor 148, identification component 145, and indicator 146 can be operably coupled to the battery, processor, and computer-readable medium of the collection device 700.

FIG. 7D is a partially schematic cross-sectional illustration of a collection device 760 including a collection reservoir 706 and an unattached cap 770, and FIG. 7E is a partially schematic cross-sectional illustration of the collection device 760 of FIG. 7D having the cap 770 attached to the collection reservoir 706. The collection device 760 is generally similar to the collection device 700 described with reference to FIGS. 7A-7C, but differs in that the collection device 760 does not include the movable switch 137. Referring to FIGS. 7D and 7E together, the collection device 760 includes a first electrically-conductive feature 756a on the cap 770 and a second electrically-conductive feature 756b on the reservoir 706. The first electrically-conductive feature 756a and the second electrically-conductive feature 756b are configured (e.g., positioned) to contact one another when the cap 770 is attached to the reservoir 706, thereby closing a circuit including the first electrically-conductive feature 756a, the second electrically-conductive feature 756b, the circuitry 139 and at least a portion of the electronic component(s) 136. Closing the circuit can operably couple a battery on the cap 770 or reservoir 706 to one or more of the electronic component(s) 136 of the collection device 760, thereby activating the electronic component(s) 136, as previously described with reference to FIGS. 7A-7C.

In some embodiments, the collection device 760 may be configured to detect, and store information associated with, whether the circuit is later opened. For example, the computer-readable medium of the collection device 760 may be configured to store information associated with when and for how long the electronic component(s) were activated. As such, a user may be able to review the stored information and determine when and for how long the circuit was open. An open circuit can correspond to the cap 770 being removed from the reservoir 706, which could provide an indication of tampering with the sample and/or contamination. Accordingly, a medical professional or researcher may use such information to determine whether a sample is usable. Furthermore, a medical professional or researcher may use the information about the open or closed circuit to help identify any potential chain-of-custody issues by identifying when and where (e.g., using the geographic location sensor of the collection device) a sample was opened.

In some embodiments, the collection devices 700, 760 can be configured to automatically establish a connection with the bodily fluid collection device 100 once the collection devices 700, 760 are activated. Stated differently, once the caps 750, 770 are attached to the respective reservoirs 106, 706 and the electronics 136 become active, a link between the collection devices 700, 760 and the device 100 used to withdraw the bodily fluid sample now contained in the collection devices 700, 760 may be automatically established. Once the link is established, the device 100 may be configured to send any characteristics of the bodily fluid samples stored on the memory of the device 100. Furthermore, once the link is established, the collection devices 700, 760 may be configured to receive the characteristics of the bodily fluid samples stored on the memory of the device 100. In doing so, the received characteristics stored on the collection devices 700, 760 can effective act as a backup of the characteristics stored on the device 100, should the device 100 become damaged.

At least one advantage of several embodiments of the present technology described in FIGS. 7A-7E is the ability to store a bodily fluid sample in a compact collection device that can also monitor and store characteristic(s) of the sample. In doing so, several embodiments of the present technology can provide additional data to the patient, medical professionals and researchers regarding quality control of the bodily fluid samples. Furthermore, since the electronic component(s) of the collection devices 700, 760 can be used to track tampering with and/or contamination of a sample, some embodiments of the present technology can be used to mitigate chain-of-custody issues with the bodily fluid sampling process.

### III. Interconnected System Including the Bodily Fluid Collection Device(s)

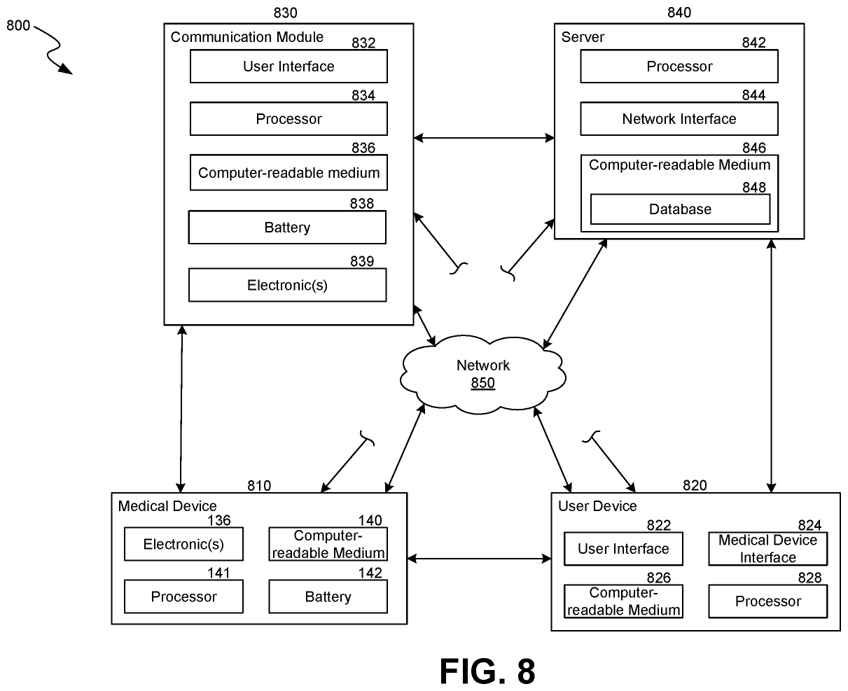

FIG. 8 is a schematic illustration of a representative interconnected computing system 800 (referred to as “the system 800”) including a medical device and configured in accordance with embodiments of the present technology. As shown in the illustrated embodiment, the system 800 can include a medical device 810, a user device 820, a communication module 830, a server 840, and a network 850 (e.g., a cloud network). The medical device 810, user device 820, communication module 830 and server 840 can be directly communicatively coupled to one another and/or indirectly communicatively coupled to one another, e.g., via the network 850. As explained in more detail below, the system 800 enables information associated with the medical device 810, patient and/or bodily fluid withdrawn from the patient to be communicated to various platforms. The system 800 can improve quality control, diagnostics and analysis, and patient satisfaction and adherence associated with the process of bodily fluid sampling.

The medical device 810 can include the device 100 previously described with reference to FIGS. 2A-2D and 4A and 4B, and/or the collection devices 700, 760 described with reference to FIGS. 7A-7E. Accordingly, the medical device 810 can comprise the electronics 136, the computer-readable medium 140 having instructions, the processor 141 configured to execute the instructions, and the battery 142 operably coupled to the electronics 136 and processor 141, e.g., after the device 810 is actuated.

The user device 820 can include a communication device, such as a mobile device (e.g., a smart phone or tablet), laptop, desktop computer, or other like device having an interface, processor and communication capabilities with an external recipient. The user device 820 can include a user interface 822 (e.g., a graphical user interface (GUI) or display), a medical device interface 824 for communicating with the medical device 810, a computer-readable medium 826 having instructions, and a processor 828 for executing the instructions of the computer-readable medium 826. The medical device interface 824 can include a mobile application, as described in additional detail with reference to FIG. 10.

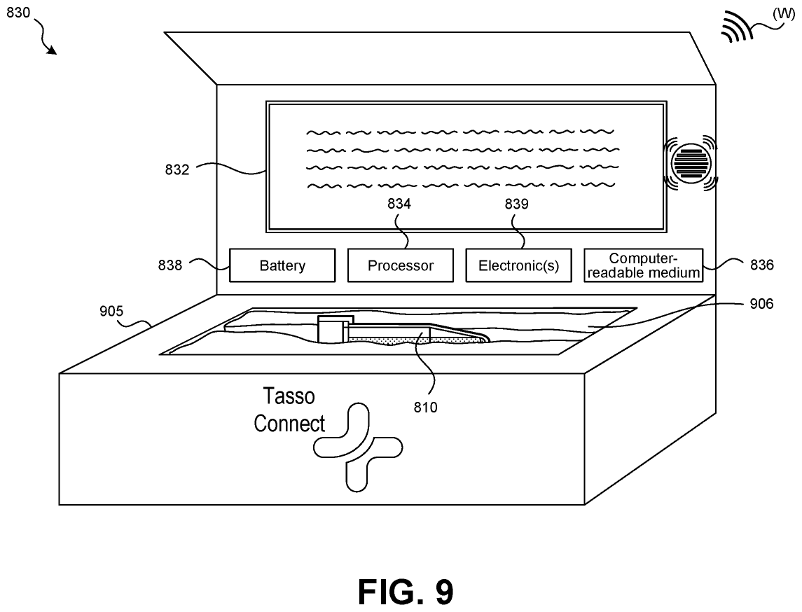

The communication module 830 can include a box or packaging configured to transport the medical device 810. As described in more detail below (e.g., with reference to FIG. 9), the communication module 830 can include a user interface 832, a processor 834, a computer-readable medium 836, and one or more electronic component(s) 839. In some embodiments, the communication module 830 can serve as an intermediary device for the medical device 810, having additional processing power and better communication capabilities than the medical device 810. In some embodiments, for example, the communication module 830 can receive any stored information on the medical device 810 and transmit that information to an external recipient, such as the user device 820 (e.g., directly or indirectly via the network 850) and/or the server 840 (e.g., directly or indirectly via the network 850). In addition to or in lieu of the foregoing, the communication module 830 can itself measure additional information via the electronic component(s) 839 onboard the communication module 830. For example, as explained in more detail below with reference to FIG. 9, the measured additional information can include real-time temperatures of the package interior, time stamps associated with a time the device was actuated, and geographic location. This measured additional information can be stored on the computer-readable medium 836 of the communication module 830 and/or transmitted to the server 840, network 850 or other external recipient, as desired.

The server 840 can include a processor 842, a network interface 844, and a computer-readable medium 846. The server 840 can use the network interface 844 to communicate with the patient, e.g., via the user device 820. The server 840 can be a Health Insurance Portability and Accountability Act (HIPAA) compliant server in that portions of the data received by the server can be anonymized prior to being stored or redistributed to other recipients. The computer-readable medium 836 can include non-volatile memory and volatile memory for storing and executing a database 838. The database 838 can receive, store and organize patient data and information recorded by individual medical devices 810. As such, the server 840 can be configured as a repository for the patient data and information recorded by individual medical devices 810. The patient data and information can include characteristics and/or symptoms of the patient and measured characteristics received from the individual medical devices 810 or bodily fluid samples. The server 840 can accumulate individual patient data and make it available to medical professionals or researchers. For example, when individual medical devices 810 are used to take bodily fluid samples for a clinical trial, the server 840 may accumulate the patient data received from the individual medical devices 810 to allow the medical professionals or researchers to characterize the data and draw conclusions therefrom.

In some embodiments, an organization may control and selectively share the information stored in the database 838. For example, a hospital may operate the server 840 and only provide access to the database 838 to physicians affiliated with or working for the hospital. Thus, the server 840 may be used to collect, store, sort, and/or organize modulation programs for patients that pay for the services of the hospital. The hospital, or other organization, may selectively provide access to the database 840 as a paid-for service to other physicians or organizations, who are not affiliated with the hospital. Additionally, in some embodiments, some organizations may control the server 840 to provide a paid-for service of providing an accumulated repository of patient data (e.g., anonymized patient data) to multiple groups or organizations. For example, an organization may enable subscribers, such as hospitals, medical groups, or individual physicians, to pay for access to the accumulated repository as part of the subscription agreement.

Advantageously, embodiments of the system 800 connect individual medical devices 810 to a computing system, thereby simplifying the transfer of data between communication devices. Additionally, the computing system 800 can provide more opportunity for the patient to interact with a medical professional and vice-versa, which in some settings has been shown to increase patient adherence, such as drug compliance. Stated differently, embodiments of the present technology can improve patient adherence and/or patient compliance regarding a particular program or regimen because the patient, medical professional, researcher, personnel associated with a clinical trial, or other interested third party can each receive an indication as to whether the patient is complying with the regimen. For example, if a compliance program dictates that a patient withdraw a bodily fluid sample at a particular time, the system 800 can be configured to automatically notify each of the parties if and/or when the patient takes a bodily fluid sample, and thus complies with the compliance program, or doesn't take a bodily fluid sample, and thus doesn't comply with the compliance program. In some embodiments, the system 800 can be configured to automatically send and/or automatically receive (a) a reminder when an upcoming action (e.g., withdrawing a bodily fluid sample) needs to be taken, and/or (b) an alert when a compliance program is or is not being followed. For example, a patient may receive a reminder when he needs to take a bodily fluid sample, and after taking the sample may then receive an indication from the system 800 (e.g., from the server 840) that the sample was successful. A successful sample may correspond to, for example, one or more measured characteristics about the bodily fluid. By issuing reminders and indications as to the status of a patient's compliance with a program, patients may be more likely to adhere to the requirements of a particular compliance program.

FIG. 9 is a partially schematic perspective view of the communication module 830 described in FIG. 8 and configured in accordance with embodiments of the present technology. The communication module 830 can include a box or package 905 configured to hold the medical device 810, and optionally a material 906 having insulative properties to aid in maintaining the withdrawn bodily fluid of the medical device 810 at or below a desired temperature. The communication module 830 can further include the user interface 832, processor 834, computer-readable medium 836, battery 838, and electronic component(s) described with reference to FIG. 8. The processor 834, computer-readable medium 836 and battery 838 can be generally identical to the processor 141, computer-readable medium 140, and battery 142, respectively, as previously described. Furthermore, the electronics 839 can be generally similar to the electronics 136 (FIGS. 2A-2D and 7A-7E), as previously described. For example, the electronics 839 can include one or more timer(s), sensor(s), identification component(s), indicator(s) and component(s) to provide communication capabilities (e.g., a transmitter, receiver, etc.). The communication components can be used to send and/or receive wireless messages (W), e.g., via one or more proprietary or standardized wireless standards, such as 802.11, Bluetooth, NFC and/or other similar communication means. The sensor(s) can include components for measuring characteristics of the communication module 830, such as temperature, time (e.g., elapsed time since an event), humidity, orientation of the medical device 810 (e.g., relative to the communication module), whether the communication module 830 is open or closed, and/or geographic location.

The medical device 810 to be stored within the communication module 830 can be electrically linked to the communication module 830. In some embodiments, for example, the medical device 810 can include an identification component (e.g., the identification component(s) 145) that can be read or scanned by the communication module 830 to establish the link (e.g., a wireless connection). Once the link is established, information already measured by the medical device 810 and stored on its computer-readable medium (e.g., the computer-readable medium 140 of FIG. 2C) may be transferred to the computer-readable medium 836 of the communication module 830. In doing so, the communication module 830 effectively creates a backup of the already measured information in case the computer-readable medium 140 becomes damaged or no longer accessible.

In some embodiments, the instructions of the computer-readable medium 836 of the communication module 830, when executed, may automatically upload any status or stored data to the server 840 (FIG. 8) or an external database. For example, the instructions of the computer-readable medium 836, when executed, may cause a wireless message to be sent from the communication module 830 to the server 840 or external database when a significant event occurs (e.g., establishing the link with the medical device 810, actuating the medical device 810, or activating the electronic component(s) on the medical device 810) and/or at predetermined time intervals (e.g., every 10 minutes).

A similar process can be performed to link the communication module 830 to the user device 820 (FIG. 8). In some embodiments, for example, the identification components of the communication module 830 can be read or scanned by the user device to establish the link (e.g., a wireless connection). Once the link is established, information on the communication module 830 may be transferred to and/or accessed by the user device 820.

The user interface 832 can include a display or GUI configured to interface with the patient. For example, in some embodiments, the processor 834 is configured to execute instructions of the computer-readable medium 836 to display informative instructions to the patient. The informative instructions can include a video and/or audio guide for, e.g., withdrawing a bodily fluid sample via the medical device 810 (e.g., the device 100 of FIGS. 2A-2D), properly sealing the medical device 810 (e.g., one of the collection devices 700, 760 of FIGS. 7A-7E), linking the communication module 830 to the medical device 810 and/or user device 820 (FIG. 8), and/or storing the medical device 810 in the communication module 830.

In some embodiments, the communication module 830 can also be used as a shipping container for transporting the medical device 810 to and/or from the patient's home or possession to the medical device manufacturer or other company. In such embodiments, the communication module 830 can measure the previously described characteristics (e.g., temperature, time, humidity, orientation, opening/closing of the communication module 830, geographic location, etc.) associated with the medical device 810 or interior of the communication module 830, and transmit the measured characteristics to the user device 820 and/or server 840.

Advantageously, the communication module 830 can enable the bodily fluid sample stored therein to be continuously monitored and tracked, e.g., via the electronic component(s) of the medical device 810 or the communication module 830. Continuous monitoring of the bodily fluid sample, e.g., for temperature and/or time elapsed since being withdrawn, can ensure quality control of the sample and reduce clinical error that currently exists with conventional processes for collecting bodily fluid samples. Additionally, tracking the communication module 830 from the time the bodily fluid sample is taken to the laboratory (or other final destination) provides a record of the chain-of-custody for the sample throughout its existence, further ensuring additional measures of quality control.

FIG. 10 is a schematic illustration of representative screenshots of a system 1000 including a communication device used to communicate with the bodily fluid collection device of FIG. 2A in accordance with embodiments of the present technology. The screenshots may be those of a communication device, such as the communication module 830 or the user device 820 previously described. As such, the screenshots may correspond to screenshots shown on the medical device interface 824 of the user device 820 (FIG. 8) or the user interface 832 of the communication module 830 (FIGS. 8 and 9). The screenshots shown in FIG. 10 generally correspond to a method for withdrawing a bodily fluid sample to be used in a clinical trial. However, similar steps of the method can be used in other applications in which patient verification is desired or necessary (e.g., for providing a bodily fluid sample for life insurance purposes).

As shown in the illustrated embodiment, the system 1000 includes a “Login” screen 1005 requesting identifying information from the user. The identifying information can serve to verify an identify of the patient and/or clinical trial the patient is participating in, and can include, amongst other required inputs, a Clinical trial ID input 1006, a Subject ID input 1007, and a Patient birthday input 1008. After entering the login information, the system verifies the information, e.g., against a database, and may link the patient to the appropriate test or trial, if not already done so. After verifying the patient's information, the system may present a “Start Sample Collection Process” screen 1010, which may present general information about the particular trial. Additionally, the screen 1010 may include a link to a “Collection Log” screen 1011 showing previous collection samples the patient has already taken. In some embodiments, the patient can click on individual previous collection samples and be provided additional information about the sample (e.g., an analysis of the sample).

After clicking the “Start Sample Collection Process” button 1012, the patient is directed to a “Prepare” screen 1015, which provides instructions to clean the patient's skin and place the device on the upper arm. Optionally, the patient can request a “Step-by-Step Video Guide” and be taken to a separate screen 1016 with multiple instructional videos for cleaning the skin in preparation for taking a sample. Here, the system 1000 may use one or more of the sensor(s) (e.g., the sensor(s) 144) of the device to identify and flag any issues associated therewith. For example, if the orientation of the device is incorrect, the system 1000 can notify the patient to adjust the orientation before proceeding. After the patient has completed preparing, the patient can click the “Next” button 1017 to verify his or her identity.

The “Verify Identity” screen 1020 asks the patient to provide identifying information, such as fingerprint information, a picture, or live video. Here, the patient may be asked to scan his or her fingerprint using a fingerprint sensor (e.g., the patient-identifying sensor 148 of FIG. 2D) on the device. The computer-readable medium of the device can cause the fingerprint information to be sent from the device to be verified, at which point an indication of the verification is sent to the communication device and the patient is directed to a subsequent step. In some embodiments, the communication device may request to access a camera, e.g., of the user device, to verify the patient via facial recognition. In addition to or in lieu of the foregoing, the clinical trial or test may require that the patient and device being used to withdraw the bodily fluid sample be simultaneously shown and verified in a frame of the camera. In doing so, the system 1000 can verify that the patient is indeed the individual taking the sample. Here, identification components (e.g., the identification component 145) of the medical device may be scanned or read by the camera to identify the particular device, and the facial features of the patient may be used to verify his or her identity. Optionally, the patient can request a “Step-by-Step Video Guide” and be taken to a separate screen 1021 with multiple instructional videos for verifying identity. Once the identity of the patient is verified, the system 1000 may automatically present a subsequent “Take a Sample” screen 1025 to the patient.

In some embodiments, patient identify must be verified before the medical device can be actuated. In such embodiments, a medical device may initially be in a locked state and only transition to an unlocked state after the patient identity is verified. For example, in such embodiments, the medical device may include a locking bar or other mechanism that prevents the actuator of the medical device from being moved toward the patient's skin. Once a patient's identity is verified, a wireless message can be sent to the medical device, enabling the medical device to then release the locking bar or mechanism, and transition the medical device from the locked state to the unlocked state.

In addition to or in lieu of the foregoing, certain electronics on the medical device may be activated after the patient identity is verified. For example, after the patient identity is verified via one or more of the mechanisms previously described, electronics used to determine orientation may be used (e.g., activated) to determine whether the device is thereafter moved beyond a predetermined threshold. Such movement may indicate, for example, that the medical device has been moved to an individual other than the patient. In such embodiments, the patient may be notified that patient identity needs to be re-verified prior to taking a bodily fluid sample.

The “Collect Sample” screen 1025 asks the patient to collect a sample by actuating the medical device. Optionally, the patient can request a “Step-by-Step Video Guide” and be taken to a separate screen 1026 with multiple instructional videos for taking a sample. In some embodiments, the system 1000 may be configured to automatically detect (e.g., via the sensor(s) 144) when a bodily fluid has been withdrawn into the medical device or reservoir. For those embodiments including the medical device interface 824 of the user device 820 (FIG. 8), the system may next direct the patient to a subsequent, “Return Package” screen 1030. For those embodiments including the user interface 832 of the communication module 830, the system may direct the patient to an “End” screen 1035.

The “Return Package” screen 1030 asks the patient to place the withdrawn sample in the return box, and to pair the return box to the user device 820 (FIG. 8). Here, the system 1000 may ask the patient to withdraw the reservoir (e.g., the reservoirs 106, 706) from the medical device and activate the collection device (e.g., the collection devices 700, 760) by securing a cap (e.g., the caps 750, 770) thereto. Optionally, the patient can request a “Step-by-Step Video Guide” and be taken to a separate screen 1031 with multiple instructional videos for properly packaging the sample and pairing the return box to the user device. Once the return package is paired with the user device, the system may direct the patient to the “End” screen 1035.

Advantageously, several embodiments of the present technology described in FIG. 10 help ensure that a bodily fluid sample is properly withdrawn. Furthermore, by providing multiple mechanisms for verifying identity of the patient prior to withdrawing a sample, the system 800 ensures that authenticity of the sample is maintained.

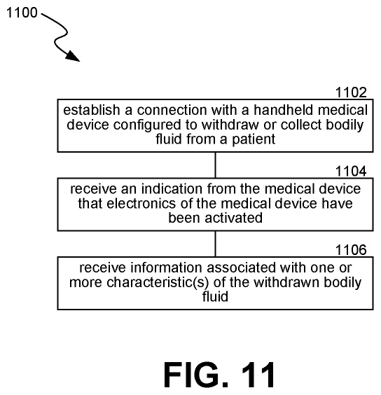

FIG. 11 is a block diagram of a method 1100 for determining characteristics associated with withdrawn bodily fluid in accordance with embodiments of the present technology. In some embodiments, the steps of the method 1100 can be performed by the user device 820, communication module 830, or server 840 previously described. As shown in the illustrated embodiment, the method 1100 includes establishing a connection with a handheld medical device configured to withdraw or collect bodily fluid from a patient (process portion 1102). In some embodiments, establishing the connection with the medical device can comprise sending a first wireless message to the medical device, and receiving a second wireless message from the medical device in response to the first message. The medical device can include the device 100 of FIG. 2A or the collection devices 700, 760 of FIGS. 7B and 7E respectively).

The method 1100 can further include receiving an indication from the medical device that electronics of the medical device have been activated (process portion 1104). In some embodiments, receiving an indication that the electronics of the medical device have been activated can be based on a circuit associated with the medical device closing.

The method 1100 can further include receiving information associated with one or more characteristic(s) of the withdrawn bodily fluid (process portion 1106). In some embodiments, receiving the information associated with the characteristic(s) can include receiving information associated with one or more sensor(s) of the medical device. For example, the information associated with the sensor(s) can include a chemical compound of the bodily fluid, a temperature of the bodily fluid, and/or a time associated with the activation of the electronics. The information associated with the chemical compound can include information regarding metabolites, biologics, biomarkers and/or proteins.

FIG. 12 is a block diagram of a method 1200 for determining one or more characteristic(s) associated with a withdrawn bodily fluid, the method 1200 including an identity verification step in accordance with embodiments of the present technology. As shown in the illustrated embodiment, the method 1200 includes establishing a connection with a handheld medical device configured to withdraw bodily fluid from a patient (process portion 1202). As previously described, establishing the connection with the medical device can comprise sending a first wireless message to the medical device, and receiving a second wireless message from the medical device in response to the first message.

The method 1200 can further include receiving information associated with an identify of the patient or identification of the medical device (process portion 1204). Information associated with the identity of the patient can include fingerprint data and/or facial recognition data received from the patient. In some embodiments, the fingerprint data can be obtained via a fingerprint sensor of the medical device, and the facial recognition data can be obtained from a communication device having an established connection with the medical device. For example, the facial recognition data may be obtained via a camera of a mobile phone. Information associated with the identification of the medical device can include data associated with an identification component of the medical device. In some embodiments, the identification component can be positioned on an outer surface of the medical device, and receiving information associated with the identification of the medical device can include receiving information associated with the identification component on the medical device.

The method 1200 can further include verifying the identifying information is associated with the patient or medical device (process portion 1206). Verifying the identifying information of the patient can include referencing a database to determine whether the identifying information provided by the patient matches patient information already stored in the database and/or is associated with the connected medical device. In some embodiments, after verifying the identifying information, the method 1200 can further comprise sending an indication to the medical device that the identifying information has been verified. The indication that the identifying information has been verified may in some embodiments enable the medical device to be actuated. Accordingly, in such embodiments, the medical device may be prevented from being actuated (e.g., by a locking mechanism) until the indication is received by the medical device.

The method 1200 can further include receiving an indication from the medical device that the medical device has been actuated (process portion 1208). In some embodiments, receiving the indication that the medical device has been actuated can include receiving an indication that one or more electronic component(s) of the medical device has been activated to an active state, based on a circuit associated with actuation of the device closing. The indication that the medical device has been actuated can correspond to an indication that bodily fluid has been withdrawn from the patient.

The method 1200 can further include receiving information associated with one or more characteristic(s) of the withdrawn bodily fluid (process portion 1210). In some embodiments, receiving the information associated with the characteristic(s) can include receiving information associated with one or more sensor(s) of the medical device. For example, the information associated with the sensor(s) can include a chemical compound of the bodily fluid, a temperature of the bodily fluid, and/or a time associated with actuation of the medical device. The information associated with the chemical compound can include metabolites, biologics, biomarkers and/or proteins.

### IV. Conclusion

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like (e.g., “including” or “having”) are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

I/We claim:

1. A device for collecting bodily fluid from a patient, the device comprising:
a housing defining a lumen and having a bottom surface configured to be positioned against skin of the patient;
a skin-piercing feature positioned at least partially within the lumen;
an electronics subsystem positioned at least partially within the housing, wherein the electronics subsystem includes a switch; and
an actuator operably coupled to the switch and the skin-piercing feature, wherein the actuator is movable through the lumen from a first position to a second position, and wherein movement of the actuator from the first position to the second position is configured to—
move the skin-piercing feature (a) through the lumen and (b) past the bottom surface of the housing to puncture the skin of the patient, to thereby draw a bodily fluid from the patient into the lumen of the housing, and
move the switch from an open position to a closed position to thereby activate the electronics subsystem.

2. The device of claim 1 wherein the electronics subsystem includes a timer configured to record a time elapsed after activation of the electronics subsystem.

3. The device of claim 1 wherein the electronics subsystem includes a sensor configured to record an identity of the patient.

4. The device of claim 3 wherein the sensor is configured to record a fingerprint of the patient.

5. The device of claim 3 wherein the sensor is configured to record an image or pattern of an eye of the user.

6. The device of claim 1 wherein the electronics subsystem includes a sensor configured to measure a characteristic of the bodily fluid.

7. The device of claim 6, further comprising a microfluidic channel positioned at least partially within the housing and configured to receive the bodily fluid, wherein the sensor is positioned at least partially within the microfluidic channel.

8. The device of claim 6, further comprising:
a microfluidic channel positioned at least partially within the housing and configured to receive the bodily fluid; and
a sample board extending between the microfluidic channel and the sensor, wherein the sample board is configured to draw a portion of the bodily fluid from the microfluidic channel to the sensor.

9. The device of claim 1 wherein the electronics subsystem includes (a) one or more processors and (b) a computer-readable medium storing instructions that, when executed by the one or more processors, cause the electronics subsystem to measure a time associated with movement of the switch from the open position to the closed position.

10. The device of claim 1 wherein the electronics subsystem includes a communication component configured to wirelessly transmit information associated with the device to an external recipient.

11. The device of claim 10 wherein the electronics subsystem includes a sensor configured to measure a characteristic of the bodily fluid, and wherein the information associated with the device includes information associated with the measured characteristic of the bodily fluid.

12. The device of claim 1 wherein the second position is nearer to the bottom surface of the housing than the first position.

13. The device of claim 1 wherein the housing is sized to be held in a hand of the patient.

14. A device for collecting bodily fluid from a patient, the device comprising:
a housing;
a skin-piercing feature positioned at least partially within the housing;
an actuator operably coupled to the skin-piercing feature, wherein the actuator is movable from a first position to a second position relative to the housing, and wherein movement of the actuator from the first position to the second position is configured to move the skin-piercing feature at least partially out of the housing to puncture the skin of the patient, to thereby draw a bodily fluid from the patient into the lumen of the housing; and
an electronics subsystem positioned at least partially within the housing, wherein the electronics subsystem includes—
a sensor configured to measure a characteristic of the bodily fluid; and
a communication component configured to wirelessly transmit information associated with the measured characteristic to an external recipient.

15. The device of claim 14 wherein the sensor is configured to measure the characteristic of the bodily fluid a predetermined time after the actuator is moved from the first position to the second position.

16. The device of claim 15 wherein the characteristic is an amount of a chemical compound in the bodily fluid.

17. The device of claim 14 wherein the electronics subsystem includes a timer configured to measure a time elapsed after the actuator is moved from the first position to the second position.

18. The device of claim 17 wherein the electronics subsystem further includes (a) a processor and (b) a computer-readable medium storing instructions that, when executed by the processor, cause the electronics subsystem to perform operations comprising—
receiving the elapsed time from the timer;
determining the predetermined time based on the elapsed time; and
controlling the sensor to measure the characteristic of the bodily fluid at the predetermined time.

19. A device for collecting bodily fluid from a patient, the device comprising:
a housing;
a skin-piercing feature positioned at least partially within the housing;
an actuator operably coupled to the skin-piercing feature, wherein the actuator is movable from a first position to a second position relative to the housing, and wherein movement of the actuator from the first position to the second position is configured to move the skin-piercing feature at least partially out of the housing to puncture the skin of the patient, to thereby draw a bodily fluid from the patient into the lumen of the housing; and
a computer-readable medium storing instructions that, when executed by one or more processors of a computing system, cause the computing system to perform operations comprising—
measuring a characteristic of the bodily fluid;
establishing a connection with an external computing device; and
communicating information associated with the measured characteristic to the external computing device.

20. The device of claim 19, further comprising a switch operably coupled to the processor, wherein movement of the actuator from the first position to the second position closes the switch to thereby activate the processor and cause the processor to execute the computer-executable instructions contained in the memory.
